(19) United States
(12) Patent Application Publication
(10) Pub. No.: US 2024/0148341 A1
(43) Pub. Date: May 9, 2024

(54) DEATH HANDLING SYSTEM

(71) Applicant: Oyekunle Afolabi, Brampton (CA)

(72) Inventor: Oyekunle Afolabi, Brampton (CA)

(21) Appl. No.: 17/982,972

(22) Filed: Nov. 8, 2022

(51) Int. Cl. A61B 5/00; A61B 5/0205; G06Q 40/08; G06Q 50/30

(52) U.S. Cl. CPC A61B 5/746; A61B 5/0004; A61B 5/02055; A61B 5/681; A61B 5/7405; A61B 5/742; G06Q 40/08; G06Q 50/30

(57) ABSTRACT

The present invention provides a system by which a deceased person is identified, and their remains are handled according to a prearrangement.

### BACKGROUND OF THE INVENTION

It is a sad reality that persons move away from family and loved ones and do not have proper identifying information upon death. In situation square governments are not able to locate next of kin, bodies are disposed of at the discretion of the local government. These disposal methods are sometimes contrary to the desires of the deceased and the family. There is a need for an automated system that will facilitate the process of notifying next of kin or designated persons.

The present invention addresses this need.

### SUMMARY OF THE INVENTION

In one embodiment, the system includes prepaid transportation for the remains to be returned to a pre-approved person and destination.

In one embodiment, a client will reach out to his insurance company for a repatriation insurance. Within the repatriation insurance the client will be assessed for risks and health to determine premiums and eligibility. Then the client will approve their arrangement or special requests of an approved concierge service and/or funeral home.

Upon approval the client will be given a bracelet to wear. This bracelet has technology within it. This technology can tell whether the user is alive or not depending on body temperature and pulse.

The bracelet will take the user's pulse by way of sensors throughout the band. The bracelet will be able to determine normal heart rates of the user through historical data as the bracelet is being worn. If the user's heart rate starts to go outside the normal range of the user, the system will become aware letting the user know that they are in danger. If this should escalate to a point where the heart stops for more than 3 minutes the bracelet will start taking the body temperature of the user as a backup to determine if the person has passed or taken the bracelet off. Should there be no pulse for 3 minutes and there is a reduced body temperature then the bracelet will determine the user has passed. The bracelet will then send an alarm to the insurance company and provide the exact location of the user. Then the insurance company will initiate all the steps of getting the body back to the country of origin. The system will prepare all paperwork, transportation and special requests made from the user to execute their wishes.

The system needs to be automated to make it as easy as possible for the family as they have just lost a member of their family and need to grieve instead of dealing with the frustrations of getting the user back to the country. This will also be a relief of the financial burden to the family as it will all be covered through the premiums of the policy.

In one embodiment, the present invention is a system for identifying and handling remains of a deceased person, said system comprising:
a central monitoring computer;
a wristband worn by a user and configured with biometric sensors and wireless communication to communicate with said central monitoring computer, said central monitoring computer monitored by a person or entity and configured to alert said person or entity when said wristband communicates a user is not alive based on information sent by said biometric sensors.

In one embodiment, the biometric sensors include measurement of pulse and body temperature.

In one embodiment, the biometric sensors include default values such that measurement of pulse and body temperature indicate death of said user.

In one embodiment, the central monitoring computer transmits contact information to an audio output, visual output, or audio and visual output to said wristband.

In one embodiment, the wristband includes printed indicia of system contact information.

In one embodiment, the system is operated by an insurance company administering a pre-paid transportation of a deceased user as pre-arranged.

In one embodiment, the wristband is configured with a GPS location chip. In a preferred embodiment, the GPS location device is activated by said central monitoring computer monitored by a person or entity. Thus, when the central monitoring computer receives information that a wearer is deceased, the system operation will then activate the GPS location device in order to ascertain the exact location of the deceased. If the deceased is in a hospital or funeral home, the system operator will then be able to contact the entity in possession of the body in order to commence the pre-selected transportation and disposal.

In one embodiment, the present invention is a system for identifying and handling remains of a deceased person, said system consisting of:
a central monitoring computer;
a wristband worn by a user and configured with biometric sensors and wireless communication to communicate with said central monitoring computer, said central monitoring computer monitored by a person or entity and configured to alert said person or entity when said wristband communicates a user is not alive based on information sent by said biometric sensors.

In one embodiment, the biometric sensors include measurement of pulse and body temperature.

In one embodiment, the invention further consists of the biometric sensors include default values such that measurement of pulse and body temperature indicate death of said user.

In one embodiment, the invention further consists of the central monitoring computer transmits contact information to an audio output, visual output, or audio and visual output to said wristband.

In one embodiment, the invention further consists of the wristband includes printed indicia of system contact information.

In one embodiment, the invention further consists of the system is operated by an insurance company administering a pre-paid transportation of a deceased user as pre-arranged.

In one embodiment, the invention further consists of the wristband is configured with a GPS location chip. In a preferred embodiment, the GPS location device is activated by said central monitoring computer monitored by a person or entity. Thus, when the central monitoring computer receives information that a wearer is deceased, the system operation will then activate the GPS location device in order to ascertain the exact location of the deceased. If the deceased is in a hospital or funeral home, the system operator will then be able to contact the entity in possession of the body in order to commence the pre-selected transportation and disposal.

### BRIEF DESCRIPTION OF THE SEVERAL VIEWS OF THE DRAWINGS

FIG. 1 is the wristband of the system according to one embodiment of the present invention.

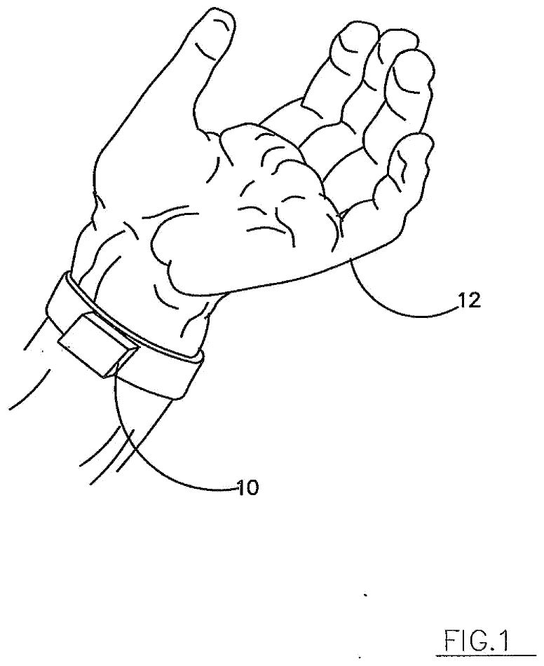

FIG. 2 is a flowchart of the system sensors according to one embodiment of the present invention.

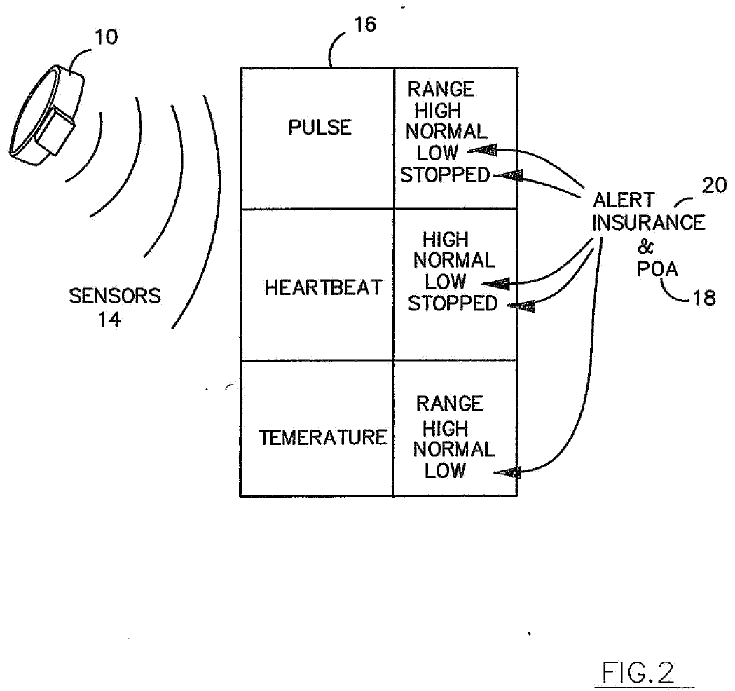

FIG. 3 is a flowchart of the system according to one embodiment of the present invention.

### DETAILED DESCRIPTION OF THE PREFERRED EMBODIMENTS

Although the drawings depict a particularized embodiment, the present invention is contemplated as configurable in any physical structure whereby the inventive scope and concept are achieved.

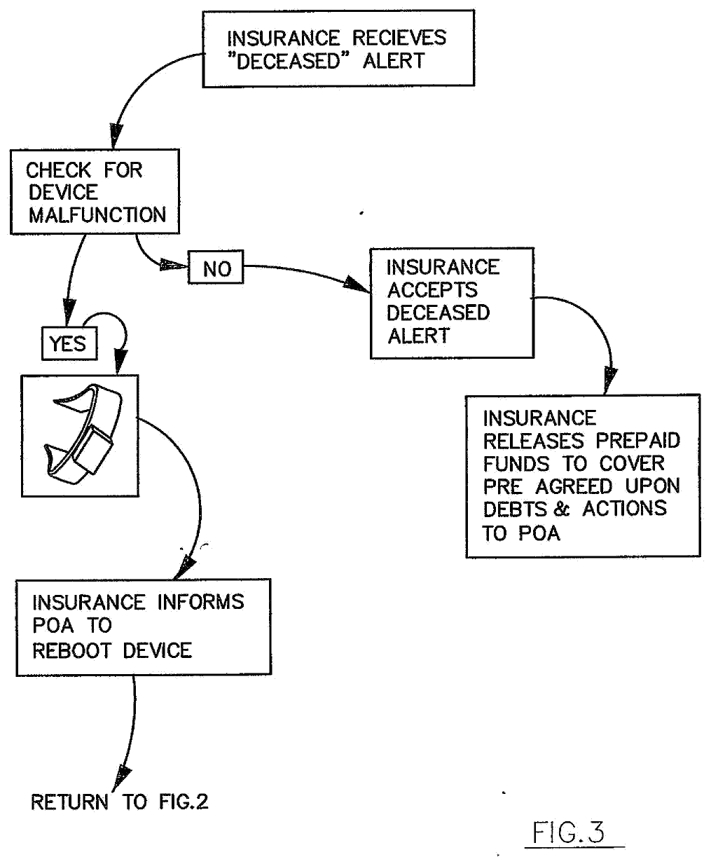

The system includes a bracelet 10 worn about the wrist of a user 12. The bracelet is constructed and arranged with sensors 14 as known in the art to measure pulse and temperature. The system is further configured with communications hardware such that if it is determined that there is no pulse and temperature has dropped to a point indicating the wearer is deceased, a wireless signal is sent to a central receiving microcomputer/microprocessor-based unit. In one environment, the receiving unit is monitored by a burial insurance entity. This is for illustration only. The central receiving unit can be monitored and operated by an independent entity. Has demonstrated in FIG. 3, when an alert is received that a where is deceased, the unit will initially perform a remote diagnostic to determine if there has been a malfunction. If a malfunction is determined, the system will reset the bracelet for continual monitoring. If there has been no malfunction detected, the system will receive the alert and initiate a process by which the remains are identified and handled as desired. Bracelet 10 is configured with printed indicia indicating contact information upon death of the wearer. In one embodiment, the bracelet has audio output, visual output, or combinations thereof, alerting with contact information. In this embodiment, a person or entity handling the remains is notified to contact a particular entity or person relating to handling up the remains.

In an environment where an insurance company has received prepaid disposition payments, the insurance company will arrange for transport of remains as pre-arranged.

While the invention has been described in its preferred form or embodiment with some degree of particularity, it is understood that this description has been given only by way of example and that numerous changes in the details of construction, fabrication, and use, including the combination and arrangement of parts, may be made without departing from the spirit and scope of the invention.

What is claimed:

1. A system for identifying and handling remains of a deceased person, said system comprising:
a central monitoring computer;
a wristband worn by a user and configured with biometric sensors and wireless communication to communicate with said central monitoring computer, said central monitoring computer monitored by a person or entity and configured to alert said person or entity when said wristband communicates a user is not alive based on information sent by said biometric sensors.

2. The system of claim 1, wherein said biometric sensors include measurement of pulse and body temperature.

3. The system of claim 1, wherein said biometric sensors include default values such that measurement of pulse and body temperature indicate death of said user.

4. The system of claim 1, wherein said central monitoring computer transmits contact information to an audio output, visual output, or audio and visual output to said wristband.

5. The system of claim 1, wherein said wristband includes printed indicia of system contact information.

6. The system of claim 1, wherein said system is operated by an insurance company administering a pre-paid transportation of a deceased user as pre-arranged.
